(19) United States
(12) Patent Application Publication
(10) Pub. No.: US 2015/0011875 A1
(43) Pub. Date: Jan. 8, 2015

(54) COMPUTED TOMOGRAPHY (CT) - HIGH INTENSITY FOCUSED ULTRASOUND (HIFU) SYSTEM AND/OR METHOD

(71) Applicant: KONINKLIJKE PHILIPS N.V., EINDHOVEN (NL)

(72) Inventors: Niels Jan Noordhoek, Best (NL); Alessandro Guido Radaelli, Oirschot (NL); Bart Carelsen, Eindhoven (NL); Nadine Abi-Jaoudeh, Washington, DC (US); Ankur Kapoor, Cranbury, NJ (US); Bradford Johns Wood, Bethesda, MD (US)

(21) Appl. No.: 14/380,418

(22) PCT Filed: Feb. 21, 2013
(86) PCT No.: PCT/IB2013/051425

§ 371 Date: Aug. 22, 2014

Related U.S. Application Data
(60) Provisional application No. 61/603,586, filed on Feb. 27, 2012.

(51) Int. Cl. A61N 7/02; A61B 6/04; A61B 8/13; A61B 6/00; A61B 5/055; A61B 6/03; A61B 19/00

(52) U.S. Cl. CPC A61N 7/02; A61B 6/032; A61B 6/0407; A61B 19/54; A61B 6/469; A61B 6/5247; A61B 6/037; A61B 5/055; A61B 8/13; A61B 19/2203; A61B 6/466; A61B 19/5244; A61N 2007/0091
USPC 600/426; 600/427

(57) ABSTRACT

A system (100) includes a CT scanner (101) with a radiation source (108) that emits radiation the traverses an examination region (112), a radiation sensitive detector array (110), located opposite the source across the examination region, which detects radiation traversing the examination region and a portion of a subject therein and generates a signal indicative thereof, and a subject support (114) that positions the subject in the examination. A HIFU apparatus (124) includes an ultrasound probe (126). A main controller (142) controls an operation of both the CT scanner and the HIFU apparatus. A treatment navigator (140) visually presents, in a graphical user interface, CT data of a location of the ultrasound probe with respect to at least one fiducial marker of the HIFU apparatus and obtained by the CT scanner overlaid over a previously generated HIFU procedure plan.

The following generally relates a computed tomography (CT)—High-Intensity Focused Ultrasound (HIFU) system and/or method.

A computed tomography (CBCT) scanner includes a radiation source and a two-dimensional radiation sensitive detector array located opposite thereof and can acquire three-dimensional (3D) volumetric data of a scanned portion of a subject in a single gantry rotation. In a C-arm arrangement, the source is located at one of the C-arm and the detector array at the other end of the C-arm, forming an examination region there between inside the “C.” The C-arm is pivotably supported and configured to pivot about a pivot point so as to position the source and detector for acquiring data at one or more different angles.

A High-Intensity Focused Ultrasound (HIFU) apparatus produces high intensity ultrasonic waves which are focused at tissue of interest (e.g., a tumor) and which heat the tissue of interest for ablation or for hyperthermia in conjunction with radiation and/or pharmaceutical (e.g., chemo) administration. Such procedures have been guided by images, which are used to plan and track the ablation or hyperthermia procedures. Magnetic resonance (MR) imaging and ultrasound (US) imaging are two imaging modalities that have been used to generate images used to plan and track ablation or hyperthermia procedures.

By way of non-limiting example, the following describes an example of pharmaceutical administration in connection with an MR imaging guided procedure. Procedures such as catheter or needle placement in a patient are often performed in interventional radiology (IR) suite. In this suite, fluoroscopy or the like is used to guide the advancement of the catheter or the needle in the patient. The patient is then transferred to an MR suite for the ablation with HIFU or the hyperthermia with HIFU during the delivery the pharmaceutical.

However, verifying the catheter or needle position and/or checking any problems would require the patient to be transferred back to the IR suite. Unfortunately, this may hinder options available to a patient. Furthermore, ablations and hyperthermia procedures can take several hours, so while the patient is in the MR suite for an ablation or hyperthermia procedure, the MR suite is unavailable for MR scanning other patients. Moreover, MR is not readily available in all hospitals and is expensive, and US does not provide suitable image resolution and quality for treatment of non-superficial lesion.

Aspects described herein address the above-referenced problems and others.

In one aspect, a system includes a CT scanner with a radiation source that emits radiation the traverses an examination region, a radiation sensitive detector array, located opposite the source across the examination region, which detects radiation traversing the examination region and a portion of a subject therein and generates a signal indicative thereof, and a subject support that positions the subject in the examination region. A HIFU apparatus includes an ultrasound probe and is physically integrated in the subject support. A main controller controls an operation of both the CT scanner and the HIFU apparatus. A treatment navigator visually presents, in a graphical user interface, CT data of a location of the ultrasound probe with respect to at least one fiducial marker of the HIFU apparatus and obtained by the scanner overlaid over a previously generated HIFU procedure plan.

In another aspect, a method includes acquiring CT image data of a HIFU ultrasound probe of a HIFU apparatus in connection with a HIFU procedure of a subject using a CT scanner, wherein the HIFU apparatus is integrated with the scanner. The method further includes overlaying the CT image data over a visual presentation of a HIFU procedure plan, which includes three dimensional volume image data and a visually identified HIFU treatment zone. The method further includes aligning the HIFU ultrasound probe with the HIFU treatment zone using the visually presented overlaid data. The method further includes visually tracking a change in a location of the HIFU ultrasound probe in the visually presented overlaid data. The method further includes performing the HIFU procedure with the aligned HIFU ultrasound probe.

In another aspect, a computer readable storage medium is encoded with computer readable instructions. The computer readable instructions, when executed by a processer, cause the processor to visually present a graphical user interface that displays CT image data, of a HIFU ultrasound probe in connection with a HIFU procedure plan for a subject, overlaid over previously acquired volumetric data of the subject, which includes graphical indicia identifying a HIFU treatment zone, in which the visual presentation visually tracks a location of the HIFU ultrasound probe with respect to the HIFU treatment zone.

The invention may take form in various components and arrangements of components, and in various steps and arrangements of steps. The drawings are only for purposes of illustrating the preferred embodiments and are not to be construed as limiting the invention.

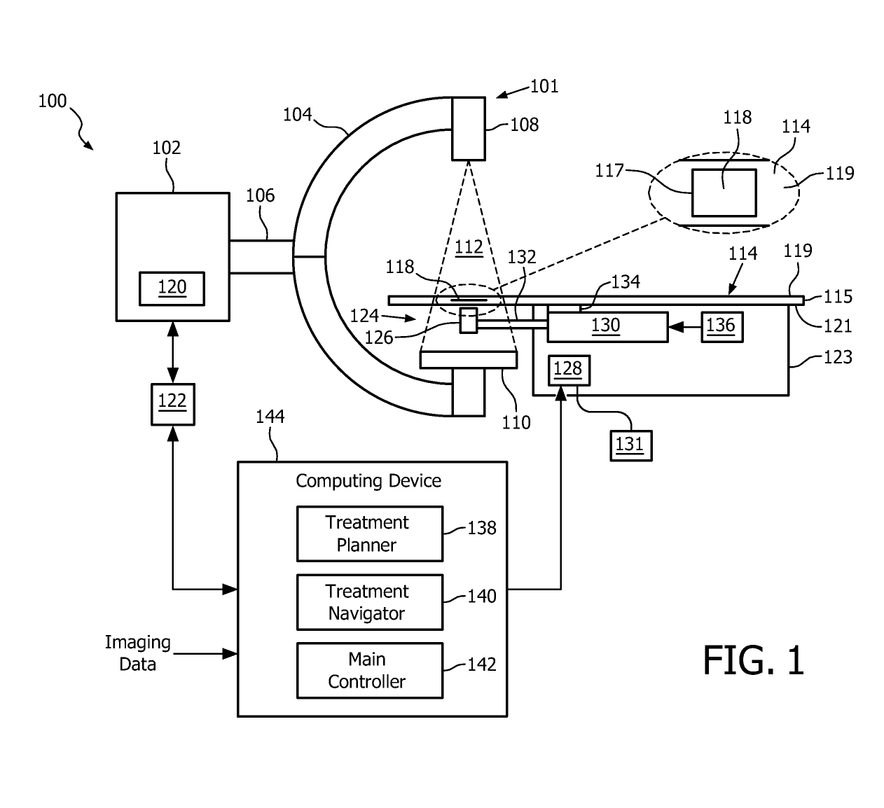

FIG. 1 schematically illustrates an example imaging system including a CT scanner with an integrated HIFU apparatus located in a subject support of the scanner.

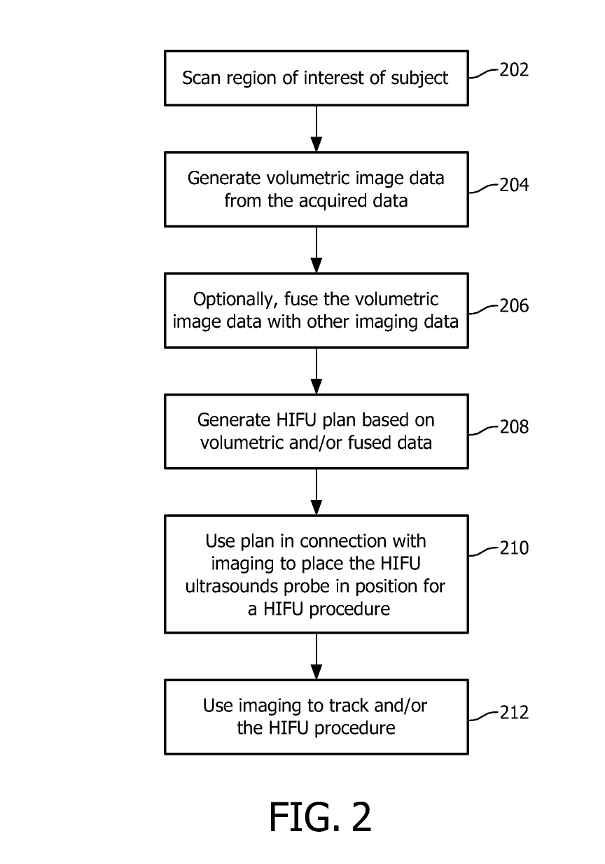

FIG. 2 illustrates an example method for guiding HIFU for ablation or hyperthermia for pharmaceutical administration using CT image data acquired by the scanner of FIG. 1.

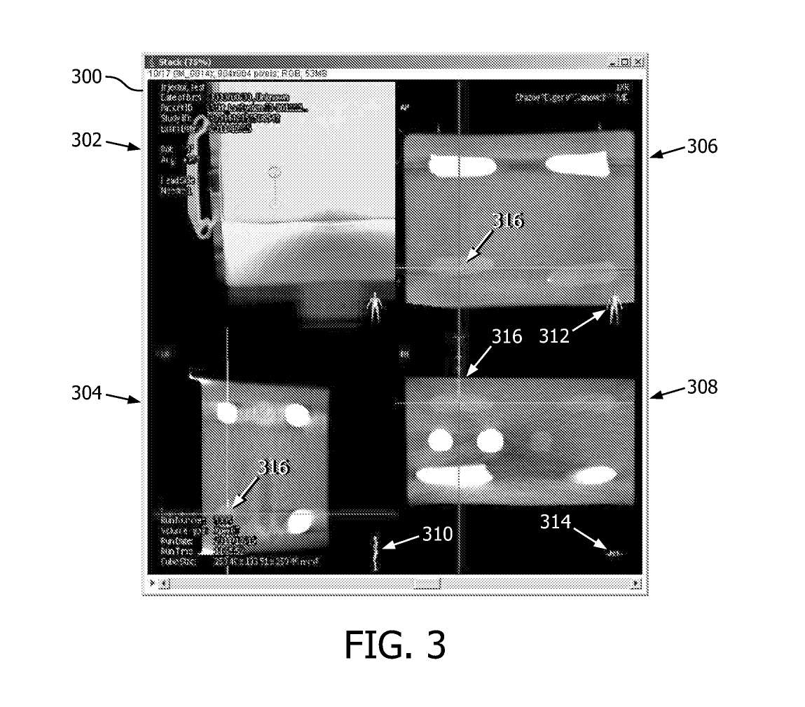

FIG. 3 illustrates an example HIFU treatment planning GUI showing volumetric image data and images of tissue of interest in images from different orientations.

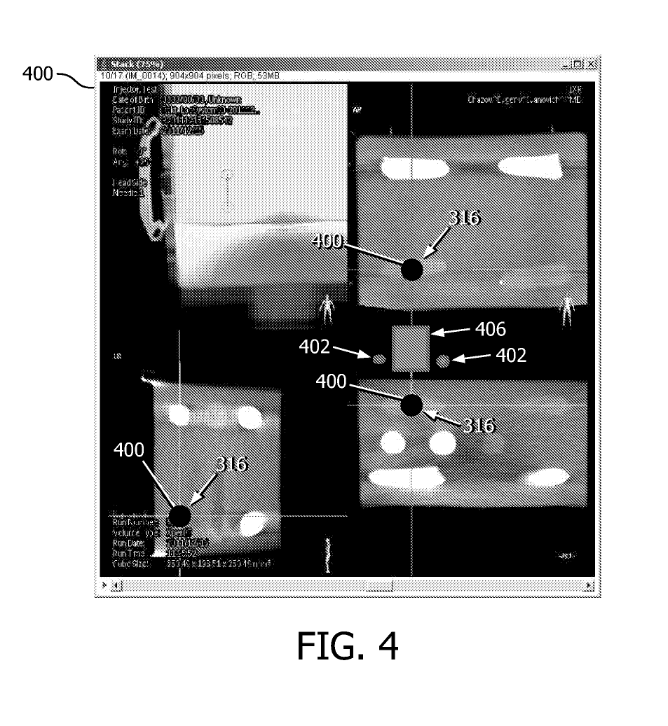

FIG. 4 illustrates the HIFU treatment planning GUI of FIG. 3 with identified treatment regions and virtual probe and markers.

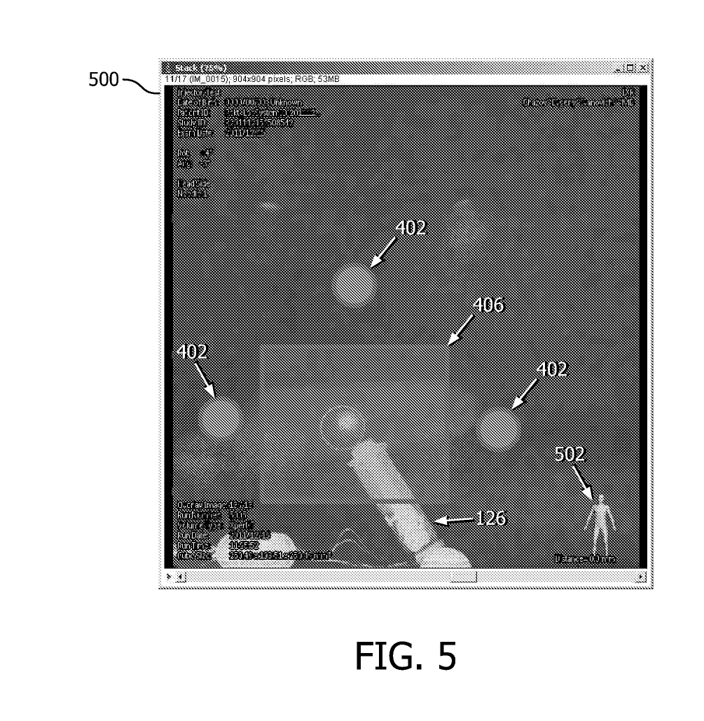

FIG. 5 illustrates a fluoroscopy image overlaid over the HIFU treatment planning GUI of FIG. 4, in which real and virtual probes and markers are aligned, from a first orientation.

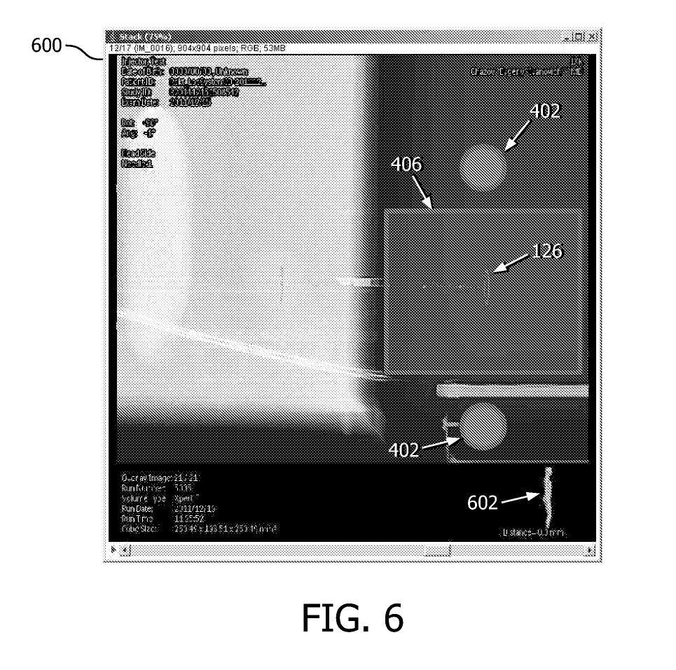

FIG. 6 illustrates a fluoroscopy image overlaid over the HIFU treatment planning GUI of FIG. 4, in which real and virtual probes and markers are aligned, from an orientation different than that of FIG. 5.

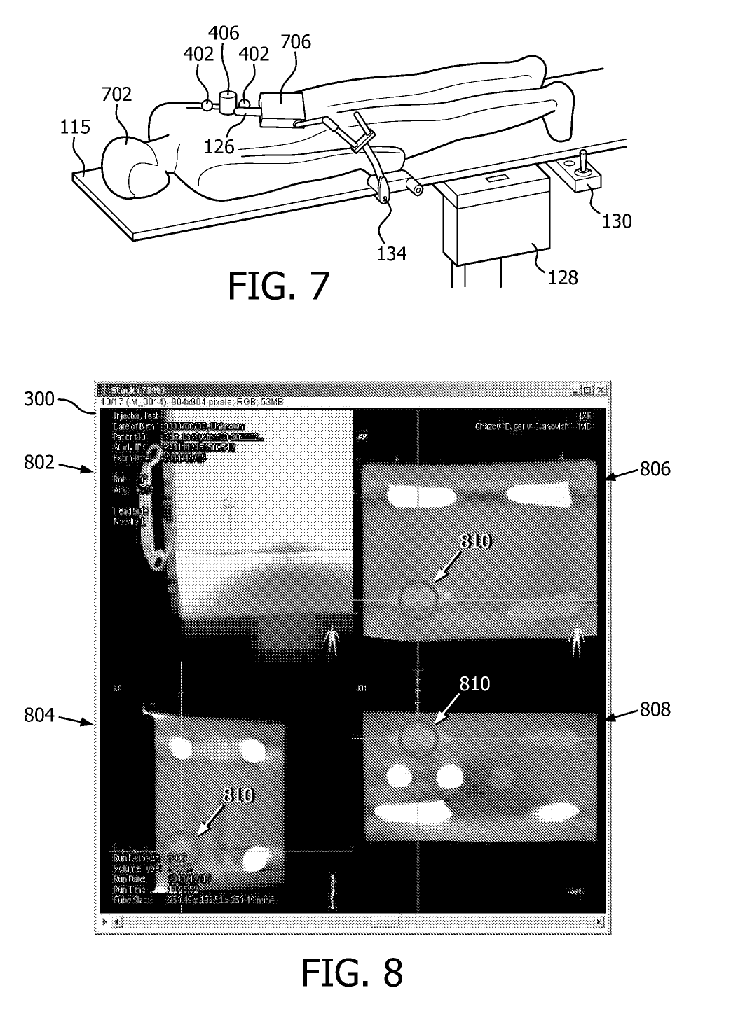

FIG. 7 schematically illustrates an example setup of the instrumentation for a HIFU procedure.

FIG. 8 illustrates a follow up HIFU GUI for verifying and/or confirming that the actual procedure maps to the plan.

FIG. 1 schematically illustrates a system 100. The system 100 includes a computed tomography (CT) scanner 101 such as a cone beam CT C-arm or other CT scanner. The scanner 101 includes stationary portion 102, which can be mounted to a ceiling, wall, floor or device in an examination room, or a portable device with wheels or the like which can be readily transported into and out of the examination room. A C-arm 104 is pivotably coupled to the stationary portion 102 via a coupling 106 and is configured to pivot through a predetermined arc (e.g., at least 180 degrees). The C-arm 104 can be pivoted before and/or during a HIFU procedure for scanning.

A radiation source 108 is coupled to one end of the C-arm 104, and a radiation sensitive detector array 110 is coupled to the other end of the C-arm 104. The radiation source 108 is separated from the detector array 110 forming an examination region 112 there between. A suitable detector array 110 includes a two-dimensional (2D) detector array such as a flat panel detector or the like. The detector array 110 generates a signal in response to detecting radiation. At least one of source 108 or the detector 110 may also move independent of the C-arm 104, for example, towards one another and/or displaced within a sleeve along the C.

A subject support 114 includes a tabletop 115 moveably affixed to a base 123. The tabletop 115 has a first side 119, which supports a subject in the examination region 112, and an opposing side 121. The illustrated tabletop 115 includes an aperture 117 that provides a material free region or opening between the sides 119 and 121. A window 118 is arrange with respect to the aperture 117 and is configured to move at least between a first position in which the window 118 closes the aperture 117 and a second position in which the window 118 does not close the aperture 117. In the illustrated embodiment, the window 118 is shown in the closed position.

The window 118 can be variously affixed to the tabletop 115. For example, in one instance, side edges of the window 118 are located within recesses of or attached to the tabletop 115 and the window 118 slides in the recesses between the at least two positions under human or machine control. An optional fastener can be used to hold the window 118 at a desired position. In another instance, the window 118 is attached to the tabletop 115 via a hinge or the like and swings between the at least two positions. Likewise, a fastener can be used to engage and hold the window 118 in an open and/or closed position, or disengaged to move the window 118 between positions. Other approaches are also contemplated herein.

A reconstructor 120 reconstructs the signal output by the detector array 110 and generates 3D volumetric image data and/or 2D images. A scanner controller 122 controls the scanner 101, including pivoting the C-arm 104 to a particular angular orientation with respect to the examination region 112, activating the source 108 to emit radiation, activating the detector array 110 to detect radiation, and receiving and/or conveying information with another device. In one instance, the scanner controller 122 also controls the window 118, for example, to open and/or close the window 118 and/or activate a mechanism (e.g., a locking pin, an electromagnetic brake, etc.) for holding the window 118 in place.

A High Intensity Focused Ultrasound (HIFU) apparatus 124 includes an ultrasound probe 126, which, in the illustrated embodiment, is positioned under the aperture 117 and the window 118 of the tabletop 115. The HIFU apparatus further includes a HIFU controller 128, which controls an operation of the probe 126 such as ultrasound transmission.

An activation device 131 such as a foot pedal, a joy stick, a remote control, or the like activates a transducer element of the probe 126 to emit high frequency focused ultrasonic waves. In the illustrated embodiment, the HIFU apparatus 124 is integrated with the scanner 101, for example, mounted in the base 123 of the subject support 114. In another embodiment, the HIFU apparatus 124 can be positioned above or to a side of the subject support 114.

A probe holder 130 includes an articulated arm 132 for positioning the probe 126 before, during and/or after a HIFU procedure. The articulated arm 132 may include one or more movable joints providing one or more degrees of freedom and may include a sub-portion that is moveable manually by a user and/or electrically via a probe holder controller 136. In the illustrated embodiment, the probe holder 130 is attached to the subject support 114 via a fastener 134. In one instance, the fastener 134 is readily removeable such as a clamp, and in another instance, the fastener 134 includes bolts, screws, rivets, or the like. In another embodiment, the probe holder 130 is affixed to a separate device such as a portable or stationary stand or the like.

A treatment planner 138 receives the reconstructed image data from the scanner 101 (and/or other scanner) and, optionally, imaging data from one or more imaging modalities such a MR, CT, US, positron emission tomography (PET), fluoroscopy, etc. The treatment planner 138 visually presents a user interactive graphical user interface (GUI) in which the received reconstructed image data and/or the optional imaging data (e.g., separately and/or fused together) are visually displayed and used to generate a HIFU plan. Such generation may include identifying one or more HIFU target zones corresponding to tissue of interest to treat and/or ultrasound probe position and/or orientation.

A treatment navigator 140 visually presents a GUI that includes data used to align and track the ultrasound probe 126, for example, in real time such that aligning and/or tracking can be concurrently performed with acquiring and presenting the data. In one instance, this includes overlaying a real-time image data (acquired by the scanner 101) of the ultrasound probe 126 over the displayed volume and aligning the probe 126 based on the generated HIFU plan. This can performed for at least two orientations, which facilitates confirming the three dimensional (3D) position of the probe 126 for a HIFU procedure. The treatment navigator 140 can also be used to display real-time image data acquired by the scanner 101 during the HIFU procedure (e.g., as discussed above by overlaying, for example, the real time data and the plan) to track the placement of the probe 126 and/or guide the ablation or hyperthermia procedure.

A main controller 142 controls at least one of the scanner controller 122, the HIFU controller 128, or the probe holder controller 136. Such control may include instructing the scanner controller 122 to pivot the C-arm 104 and/or activate the radiation source 108 and the radiation sensitive detector array 110. Such control may include instructing the HIFU controller 128 to emit high intensity focused ultrasonic waves concentrated at the tissue of interest. Such control may include instructing the probe holder controller 136 to position the probe 126 under the aperture 117 and connection with the HIFU markers for a HIFU procedure and/or move the probe 126 to a home or storage location in subject support 114.

The treatment planner 138, the treatment navigator 140 and/or the main controller 142 can be implemented via one or more processors (micro-processors, controllers, etc.) executing one or more computer executable instructions embedded or encoded on computer readable storage medium such as physical memory or other non-transitory storage medium. Additionally or alternatively, the one or more processors can execute a computer executable instruction carried by a signal, carrier wave, and/or other transitory storage medium. In the illustrated embodiment, the treatment planner 138, the treatment navigator 140 and the main controller 142 are included in computer readable storage medium of a computing device 144. In another embodiment, one or more of the treatment planner 138, the treatment navigator 140 or the main controller 142 may be otherwise located.

In a variation of the above, the scanner 101 is a conventional closed ring (donut hole) shaped CT scanner.

In another variation, the HIFU apparatus 124 is located on a cart or other device outside of the base 123 of the subject support 114. In this instance, the HIFU apparatus 124 can be used through the aperture 117 of the subject support 114 as described above, or from above or to the side of the subject support 114. Where the HIFU apparatus 124 is not used from below the tabletop 115, the aperture 117 and the window 118 can be omitted.

In another variation, the HIFU apparatus 124 is located and stored above the subject in connection with the scanner 101 or otherwise Likewise, in this instance, the HIFU apparatus 124 can be used through the aperture 117 of the subject support 114, or from above or to the side of the subject support 114, and where the HIFU apparatus 124 is not used from under the tabletop 115, the aperture 117 and the window 118 can be omitted.

In another variation, the probe 126 can additionally or alternatively be tracked using optical and/or electro-magnetic (EM) tracking.

In another variation, the probe 126 includes a component (e.g., a thermistor, etc.) that measures a value indicative of a temperature of the region of interest.

FIG. 2 illustrates a non-limiting method.

The method is for guiding a HIFU procedure such as an ablation or hyperthermia for pharmaceutical administration. The guidance is performed using CT image data acquired by the scanner 101 or conventional CT scanner. In this example, the HIFU apparatus (e.g., the HIFU apparatus 124) is physically integrated with the scanner 101.

It is to be appreciated that the ordering of the acts in the methods described herein is not limiting. As such, other orderings are contemplated herein. In addition, one or more acts may be omitted and/or one or more additional acts may be included.

At 202, a region of interest of a subject is scanned.

At 204, the acquired data is reconstructed, generating volumetric image data.

At 206, the volumetric image data is optionally fused with other imaging data.

At 208, the volumetric image data (and/or fused data) is used to generate a HIFU procedure plan. As described herein, this includes identifying one or more HIFU zones and/or probe locations and/or orientations based on the HIFU procedure plan.

At 210, the plan is used in connection with, for example, real time imaging to place the HIFU ultrasound probe in position for the HIFU procedure based on the plan.

At 212, imaging with the CT scanner is used to track and/or guide the HIFU procedure.

At least a portion of the above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

Example workflow using the system 100 described herein is discussed next in connection with FIGS. 3-8.

Initially referring to FIG. 3, the treatment planner 138 displays a GUI 300, which visually presents a volume of image data at 302 and selected images 304, 306 and 308 from the volume image data from different orientations 310, 312 and 314, all showing target tissue of interest 316.

In FIG. 4, a user identified planned ablation zone 400 is shown in the GUI 300 in connection with the different images 304, 306 and 308. Also shown in the GUI 300 are graphical indicia representing x-ray opaque markers 402 and a virtual HIFU probe 406.

In FIGS. 5 and 6, the treatment navigator 140 displays GUIs 500 and 600. In FIG. 5, the GUI 500 shows a front orientation 502 with a real time image obtained by the scanner 101 overlaid over the volume of image data with the HIFU ultrasound probe 126 and markers aligned with the virtual probe 406 and the markers 402. FIG. 6 shows that same information but from a different orientation 602.

FIG. 7 shows a subject 702 lying on the tabletop 115, along with the HIFU apparatus 124, including the probe 126, the probe controller 128, the probe activator 131 (a joy stick in this embodiment), the articulated arm 132 and the fastener 134, and virtual probe 406 and markers 402. The articulated arm 132 includes a clamp 704, which is manually positioned, and a robotic arm 706, which is electronically positioned via the probe activator 131. Note that in this embodiment, the probe 126 is positioned above the subject 702 and not under the tabletop 115 behind the aperture 117.

FIG. 8 shows the GUI 300 with data from a follow scan, including follow up volumetric data 802, and follow up images 804-808. The images in this GUI can be used to verify and confirm that the actual ablation 810 aligns with the plan.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system comprising:
a CT scanner, including:
a radiation source configured to emit radiation the traverses an examination region;
a radiation sensitive detector array, located opposite the source across the examination region, configured to detect radiation traversing the examination region and generates a signal indicative thereof; and
a subject support including a base;
a HIFU apparatus including an ultrasound probe, wherein the HIFU apparatus is integrated in the base of the subject support;
a main controller configured to control an operation of both the CT scanner and the HIFU apparatus; and
a treatment navigator configured to visually present, in a graphical user interface, CT image data, showing a location of the ultrasound probe with respect to at least one fiducial marker for a HIFU procedure wherein the CT image data is acquired by the scanner, overlaid over a previously generated HIFU procedure plan.

2. The system of claim 1, wherein a change in a position of the ultrasound probe with respect to the at least one fiducial marker is visually tracked in the visual presentation of the CT data overlaid over the previously generated HIFU procedure plan image.

3. The system of claim 1, further comprising:
a treatment planner configured to visually present in a user interactive graphical user interface previously obtained CT volume data, receives a signal indicative of a user identified HIFU procedure zone, and generates the HIFU procedure plan based on the previously obtained CT volume data and the HIFU procedure zone.

4. The system of claim 3, wherein the previously obtained CT volume data is fused with a MR, PET or US data, and the HIFU procedure plan is generated based on the combined data and the HIFU procedure zone.

5. The system of claim 1, wherein the HIFU apparatus is integrated in a base of the subject support, and the subject support, comprising:
an aperture; and
a window configured to move between a first position in which the aperture is closed and a second position in which the aperture is open,
wherein the ultrasound probe is positioned behind the aperture and is activated to apply HIFU ultrasonic waves during the HIFU procedure when the window is in the second position.

6. The system of claim 5, further comprising:
a probe holder affixed to the subject support, wherein the probe holder holds the ultrasound probe in position behind the aperture.

7. The system of claim 6, the probe holder, comprising:
an articulated arm configured to move between at least two positions, wherein moving the articulated arm between the at least two positions arm moves the probe between the at least two positions.

8. The system of claim 6, further comprising:
a probe holder controller configured to electronically moves the articulated arm between the at least two positions.

9. The system of claim 1, wherein the HIFU procedure is a procedure from a group of procedures consisting of a tissue ablation procedure or tissue hyperthermia procedure in connection with pharmaceutical administration to tissue of interest.

10. The system of claim 1, wherein the HIFU apparatus is physically integrated in the subject support.

11. The system of claim 1, wherein the treatment navigator visually presents real time CT data in the graphical user interface in real time overlaid over the previously generated HIFU procedure plan.

12. A method, comprising:
acquiring CT image data of a HIFU ultrasound probe of a HIFU apparatus in connection with a HIFU procedure using a CT scanner;
overlaying the CT image data over a visual presentation of a HIFU procedure plan, which includes three dimensional volume image data and a visually identified HIFU treatment zone;
aligning the HIFU ultrasound probe with the HIFU treatment zone using the visually presented overlaid data, wherein the HIFU ultrasound probe is part of a HIFU apparatus integrated into a base of a subject support of the CT scanner; and
visually tracking a change in a location of the HIFU ultrasound probe in the visually presented overlaid data.

13. The method of claim 12, further comprising:
obtaining three dimensional volume image data;
displaying the obtained three dimensional volume image data in a graphical user interface;
receiving a signal indicative of a user identified location of the HIFU treatment zone in the obtained three dimensional volume image data; and
generating the HIFU procedure plan based on the obtained three dimensional volume image data and the signal.

14. The method of claim 13, further comprising:
fusing the obtained three dimensional volume image data with previously acquired imaging data;
displaying the fused data in a graphical user interface;
receiving a signal indicative of a user identified location of the HIFU treatment zone in the fused data; and
generating the HIFU procedure plan based on the fused data and the signal.

15. The method of claim 12, further comprising:
acquiring second CT image data during the HIFU procedure;
overlaying the second CT image data over the visual presentation of a HIFU procedure plan; and
tracking the HIFU procedure based on the visual presentation of the second CT image data overlaid over the HIFU procedure plan.

16. The method of claim 12, further comprising:
acquiring third CT image data during the HIFU procedure;
overlaying the third CT image data over the visual presentation of a HIFU procedure plan; and
verifying correct placement of HIFU ultrasound probe for the HIFU procedure based on the visual presentation of the third CT image data overlaid over the HIFU procedure plan.

17. The method of claim 12, further comprising:
acquiring fourth CT image data during the HIFU procedure;
overlaying the fourth CT image data over the visual presentation of a HIFU procedure plan; and
determining an effectiveness of the HIFU procedure based on the visual presentation of the fourth CT image data overlaid over the HIFU procedure plan.

18. The method of claim 12, further comprising:
opening a window covering an aperture of a tabletop of the subject support, wherein the HIFU ultrasound probe is located under the aperture; and
aligning the HIFU ultrasound probe with the HIFU treatment zone for treatment of the subject through the aperture with the window open.

19. The method of claim 18, further comprising:
closing the window., thereby covering the aperture, when not performing the HIFU procedure.

20. The method of claim 18, further comprising:
moving an articulated arm of the HIFU apparatus supporting the HIFU ultrasound probe so as to move the HIFU ultrasound probe outside of the aperture.

21. A computer readable storage medium encoded with computer readable instructions, which, when executed by a processor, causer the processor to visually present of a graphical user interface that displays CT image data, of a HIFU ultrasound probe in connection with a HIFU procedure plan, overlaid over previously acquired volumetric data, which includes graphical indicia identifying a HIFU treatment zone, in which the visual presentation visually tracks a location of the HIFU ultrasound probe with respect to the HIFU treatment zone.
